(19) United States
(12) Patent Application Publication
(10) Pub. No.: US 2011/0117546 A1
(43) Pub. Date: May 19, 2011

(54) INCREASE OF SIGNAL SENSITIVITY USING DUAL PROBES IN PCR REACTIONS

(75) Inventors: Shanavaz L. Nasarabadi, Livermore, CA (US); Max Sanchez, San Jose, CA (US)

(73) Assignee: MICROFLUIDIC SYSTEMS, INC., Fremont, CA (US)

(21) Appl. No.: 12/621,367

(22) Filed: Nov. 18, 2009

(51) Int. Cl. C12Q 1/68

(52) U.S. Cl. 435/6

(57) ABSTRACT

A method increases the signal strength generated when performing real-time PCR on a target nucleic acid sequence. The method performs real-time PCR using forward primers, forward probes, reverse primers, reverse probes, nucleotides for strand/antistrand extension, and nucleic acid polymerase. Two different types of probes are used, a forward probe configured to anneal to a sense strand of a target nucleic acid sequence and a reverse probe configured to anneal to an antisense strand of the target nucleic acid sequence. The forward probe is complementary to an inner sequence of the target sense strand, and the reverse probe is complementary to an inner sequence of the target antisense strand. The forward probe and the reverse probe each include the same detectable element, that when released from the probe during strand extension results in an additive detectable signal.

### GOVERNMENT LICENSE RIGHTS

This invention was made with Government support under Agreement No. W81XWH-04-9-0010 awarded by the Government. The Government has certain rights in this invention.

### FIELD OF THE INVENTION

The invention relates to a method of performing nucleic acid amplification. More particularly, the invention relates to a method of increasing signal sensitivity using dual probes in nucleic acid amplification reactions.

### BACKGROUND OF THE INVENTION

Polymerase chain reaction (PCR) is a molecular technique for enzymatically replicating specific nucleic acid sequences, or target sequences. In particular, PCR is used to amplify relatively short, well-defined polynucleotide sequences within a given target sequence. A specific target sequence to be amplified is determined by selecting primers. Primers are short, artificial strands, often not more than fifty and usually only 18 to 25 nucleotides long that are complementary to a starting point of the specific target sequence to be amplified. The primer anneals to the target sequence at the starting point and begins the synthesis of the new target sequence through nucleic acid polymerization and hybridization. A first end of a polynucleotide is referred to as a 3′ end, and a second end of a polynucleotide is referred to as a 5′ end. The primer binds to the 3′ end of the nucleic acid target sequence.

The objective is to detect if one or more specific target sequences are present within a sample solution. The target sequence to be detected is double-stranded DNA or RNA that includes a sense strand and an antisense strand, as shown in FIG. 1. Nucleic acid polymerase and primers specific to the target sequence to be amplified are added to the sample solution. The primers are typically configured as primer pairs, a forward primer of the primer pair is an oligonucleotide that is complementary to the 3′end of the sense strand of the nucleic acid target sequence, and a reverse primer of the primer pair is an oligonucleotide that is complementary to the 3′ end of the antisense strand of the nucleic acid target sequence. In some applications, amplification is performed using thermal cycling, which includes cycles of repeated heating and cooling for nucleic acid melting and enzymatic replication of the nucleic acid. The heating step is also referred to as a denaturation step. The cooling step is also referred to as an annealing and extension step. The polymerase is heat-stable within the temperature range of the thermal cycling.

The primary function of a polymerase is the polymerization of a new nucleic acid sequence against an existing nucleic acid template. Nucleic acid polymerization is the process by which two nucleotides are bound together to form a chain. Polymerase catalyzes the synthesis of a polynucleotide sequence against a nucleotide template strand using base-pairing interactions. The polymerase enzymatically assembles the new nucleic acid strand from nucleotide building blocks using the single-stranded nucleic acid as the template and the primer, where the primer initiates the nucleic acid synthesis. The selectivity of the amplification process results from the use of primers that are complementary to the nucleic acid sequence targeted for amplification.

The denaturation step causes separation of the template nucleic acid sequence (first strand) and the primer/synthesized nucleic acid sequence (second strand) by disrupting the hydrogen bonds between complementary bases of the two strands, yielding single strands of nucleic acid sequences that are each subsequently used as templates. FIG. 2 illustrates denatured sense strand and antisense strand of the target sequence of FIG. 1. The annealing step enables annealing of the primers to the single-stranded nucleic acid templates, as shown in FIG. 3. Polymerase binds to the primer and template and begins nucleic acid synthesis. The extension step continues and completes the nucleic acid synthesis started during the annealing step, as shown in FIG. 4. The polymerase synthesizes a new strand complementary to the template strand by adding nucleotide bases, for example deoxynucleoside triphosphates (dNTPs), that are complementary to the template in a 5′ end to 3° end direction of the strand being synthesized. The synthesis is accomplished by condensing the 5′ end of the dNTPs, for example, with the 3′ end of the nucleic acid strand being extended. The extension time depends both on the polymerase used and on the length of the target sequence being amplified.

Once the thermal cycling amplification is completed, it is determined if the target sequence is present using conventional detection techniques including gel electrophoresis or application of detection chemistries, such as the use of a labeled probe with the amplified nucleic acid in a hybridization assay.

Real-time PCR enables the real time measurement of nucleic acid target sequence amplification during PCR. Detection chemistries, such as probes, are added to the polymerase and the primers prior to thermal cycling. A probe is configured to bond with an inner sequence within the target sequence, where the inner sequence is considered “downstream” of the primer binding site. As the target sequence is replicated, an element within the probe is detached. The element is not detectable when attached to the probe, but is detectable when separated from the probe. An exemplary technique utilizes probes that includes a fluorescent “reporter” molecule and a quencher molecule, such as a Taqman® probe. As part of the probe, whenever the reporter molecule is excited, the resulting released excitation energy is absorbed by the quencher molecule, where the energy is either dissipated or emitted at a different emission frequency than that of the reporter molecule. During amplification, the reporter molecule is cleaved from the probe, thereby releasing the reporter molecule for detection.

The probe anneals to the inner sequence within the target sequence, the inner sequence being in the direction of extension, referred to as “downstream”, of a primer binding site. During strand extension by a nucleic acid polymerase, such as Taq (Thennus aquaticus bacterium) polymerase, the annealed probe is digested by the 5′ to 3′ exonuclease activity of the polymerase, thereby cleaving the reporter molecule from the probe. Upon separation from the probe, the reporter molecule is no longer close enough to the quencher molecule for the quencher molecule to quench emissions from the reporter molecule by energy transfer. As amplification continues, more and more reporter molecules are cleaved from the probes, thereby increasing the fluorescent signal to be detected. The amount of fluorescence detected is directly proportional to the amount of released reporter molecules and the amount of the target sequence present.

FIGS. 5-7 illustrate a conventional real-time nucleic acid amplification and detection process using PCR. A sense strand and an antisense strand of a target nucleic acid sequence to be amplified are shown. During each thermal cycle of the nucleic acid amplification process, the sense and antisense strands are separated during the heating, or denaturing, step of the cycle, as shown in FIG. 2. The separated strands then function as templates during the subsequent cooling, or annealing, step of the thermal cycle. Prior to the thermal cycling steps, PCR reagents are added to the target sequence to be amplified. The PCR reagents include primer pairs, probes, and polymerase within a buffer solution. Each primer pair includes a forward primer and a reverse primer. The forward primer is complementary to the 3′ end of the sense strand, and therefore anneals to the 3′ end of the sense strand during the annealing step, as shown in FIG. 3. Similarly, the reverse primer is complementary to the 3° end of the antisense strand, and therefore anneals to the 3′ end of the antisense strand during the annealing step, as shown in FIG. 3. Each probe is an oligonucleotide including a reporter molecule on the 5′ end and a quencher molecule on the 3° end of the probe. Each probe is complementary to an inner sequence of the sense strand. During the annealing step, a probe anneals to the inner sequence of the sense strand, which is downstream, e.g. in the direction of strand extension, from the annealed forward primer, as shown in FIG. 5. While both the reporter molecule and the quencher molecule remain bound to the probe, either in solution or annealed to the sense strand, the reporter molecule is not detectable because of the proximity of the quencher molecule.

During an extension step of the thermal cycle, a 5′ end to 3′ end strand extension, or polymerization, of both the sense strand and the antisense strand is performed. FIG. 5 illustrates the sense strand and the antisense strand at the onset of polymerization. Strand extension begins from the forward primer annealed to the 3° end of the sense strand and progresses toward the 5′ end of the sense strand. Similar strand extension begins on the antisense strand beginning from the reverse primer annealed to the 3′ end of the antisense strand.

During strand extension, the probe annealed to the sense strand is digested by the 5′ end to 3′ end nuclease activity of the polymerase. FIG. 6 illustrates the 5′ end of the probe being cleaved, thereby releasing the reporter molecule. FIG. 7 illustrates completion of the polymerization process including digestion of the entire probe annealed to the sense strand. Upon digestion, the quencher molecule is no longer close enough to the reporter molecule to quench emissions by energy transfer. As more and more probes are digested during the thermal cycling amplification process, a stronger and stronger fluorescent signal is generated.

A number of thermal cycles must be performed before a sufficiently strong signal is generated for detection. Reducing the number of thermal cycles needed for detection is desirable. Examples of nucleic acid amplification and detection using PCR and probes is found in U.S. Pat. No. 4,683,195, U.S. Pat. No. 5,210,015, and U.S. Pat. No. 5,538,848, which are hereby incorporated in their entirety by reference.

Further, there is a limit of detection based on the amount of the original nucleic acid sample. Such restrictions minimize if not eliminate the effectiveness of PCR when used in applications having low copy targets, such as during early viral or bacterial infections where the rate of infection is trying to be measured. Increasing the limit of detection is therefore also desirable.

### SUMMARY OF THE INVENTION

An amplification and detection method increases the signal strength generated when performing real-time PCR on a target nucleic acid sequence. The amplification and detection method performs real-time PCR using forward primers, forward probes, reverse primers, reverse probes, nucleotides for strand/antistrand extension, and nucleic acid polymerase. Two different types of probes are used, a forward probe configured to anneal to a sense strand of a target nucleic acid sequence and a reverse probe configured to anneal to an antisense strand of the target nucleic acid sequence. The forward probe is complementary to an inner sequence of the target sense strand, and the reverse probe is complementary to an inner sequence of the target antisense strand. The forward probe and the reverse probe each include the same detectable element that when released from the probe during strand extension results in a detectable signal. Since the forward probe and the reverse probe use the same detectable element, the signal resulting from the detectable element being released from the sense strand is additive to the signal resulting from the detectable element being released from the antisense strand.

In one aspect, a method of detecting nucleic acid amplification is disclosed. The method includes performing a thermal cycling process on a fluid sample including a target nucleic acid sequence to be amplified, nucleic acid polymerase, a forward primer, a forward probe, a reverse primer, and a reverse probe, wherein during an annealing step of the thermal cycling process the forward primer is configured to anneal to a first complementary sequence of a sense strand of the target nucleic acid sequence, the forward probe is configured to anneal to a second complementary sequence of the sense strand, the reverse primer is configured to anneal to a first complementary sequence of an antisense strand of the target nucleic acid sequence, and the reverse probe is configured to anneal to a second complementary sequence of the antisense strand. The forward probe includes a detectable element that is not detectable when attached to the forward probe and is detectable when separated from the forward probe. The reverse probe includes the detectable element that is not detectable when attached to the reverse probe and is detectable when separated from the reverse probe. During an annealing step of the thermal cycling process, a strand extension is performed on the sense strand and the antisense strand using the nucleic acid polymerase, thereby cleaving the forward probe and the reverse probe which separates the detectable element from the forward probe and the detectable element from the reverse probe. The method also includes detecting the separated detectable elements.

In some embodiments, the first complementary sequence of the sense strand is a 3′ end of the sense strand, and the first complementary sequence of the antisense strand is a 3′ end of the antisense strand. The first complementary sequence of the sense strand and the second complementary sequence of the sense strand can be completely separate sequences, or the first complementary sequence of the sense strand and the second complementary sequence of the sense strand can partially overlap. The first complementary sequence of the antisense strand and the second complementary sequence of the antisense strand can be completely separate sequences, or the first complementary sequence of the antisense strand and the second complementary sequence of the antisense strand can partially overlap.

In some embodiments, the forward probe further includes a suppressing element that suppresses detection of the detectable element when both the detectable element and the suppressing element are attached to the forward probe, and the reverse probe further includes the suppressing element that suppresses detection of the detectable element when both the detectable element and the suppressing element are attached to the reverse probe. The detectable elements can be a reporter molecule and the suppressing element can be a quencher molecule.

In some embodiments, the strand extension is a 5′ end to 3′ end strand extension. In some embodiments, the second complementary sequence of the sense strand is different than the second complementary sequence of the antisense strand. An original sample of the target nucleic acid sequence can be single-stranded or double-stranded. In some embodiments, performing the thermal cycling process includes performing a polymerase chain reaction amplification process.

### BRIEF DESCRIPTION OF THE DRAWINGS

The accompanying drawings, which are incorporated in and form a part of this specification, illustrate embodiments of the amplification and detection method and, together with the description, serve to explain the principles of the sample preparation method but not limit the sample preparation method to the disclosed examples.

FIG. 1 illustrates a double-stranded target nucleic acid sequence including a sense strand and an antisense strand bonded to each other.

FIG. 2 illustrates denatured sense strand and antisense strand of the target nucleic acid sequence of FIG. 1.

FIG. 3 illustrates an annealing step performed during a conventional amplification process using PCR.

FIG. 4 illustrates an extension step performed during a conventional amplification process using PCR.

FIGS. 5-7 illustrate a conventional nucleic acid amplification and detection process using PCR.

FIGS. 8-10 illustrate a nucleic acid amplification and detection method using PCR.

FIG. 11 illustrates signal strength test results measured at the end of PCR for a first exemplary single-plex target nucleic acid sequence.

FIG. 12 illustrates signal strength test results measured at the end of PCR for a second exemplary single-plex target nucleic acid sequence.

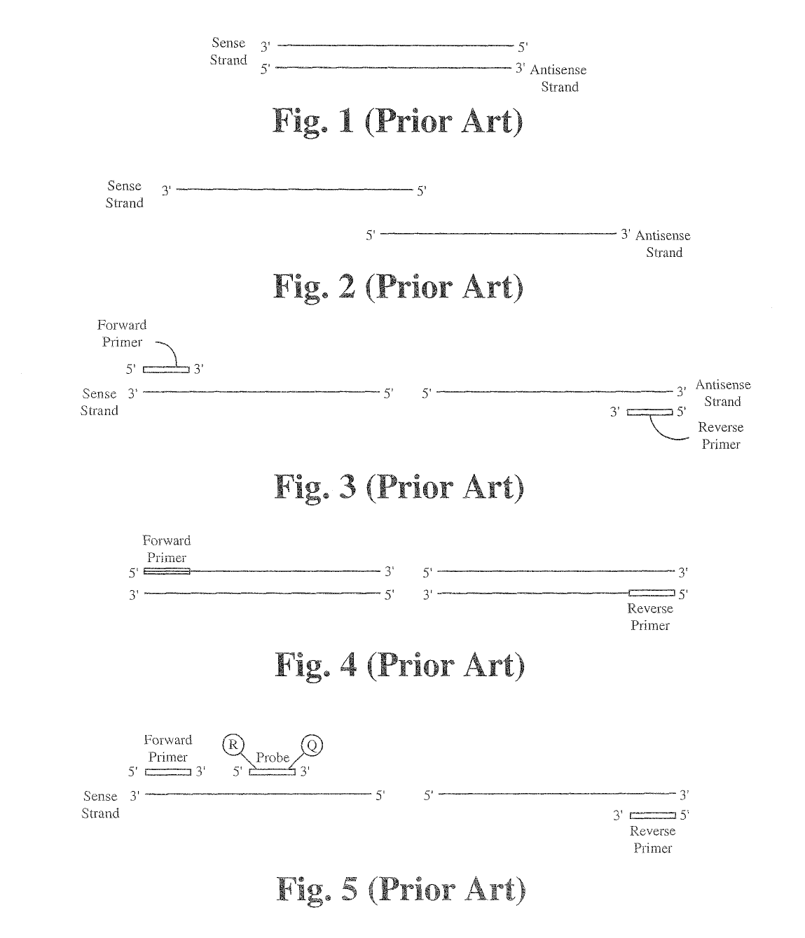

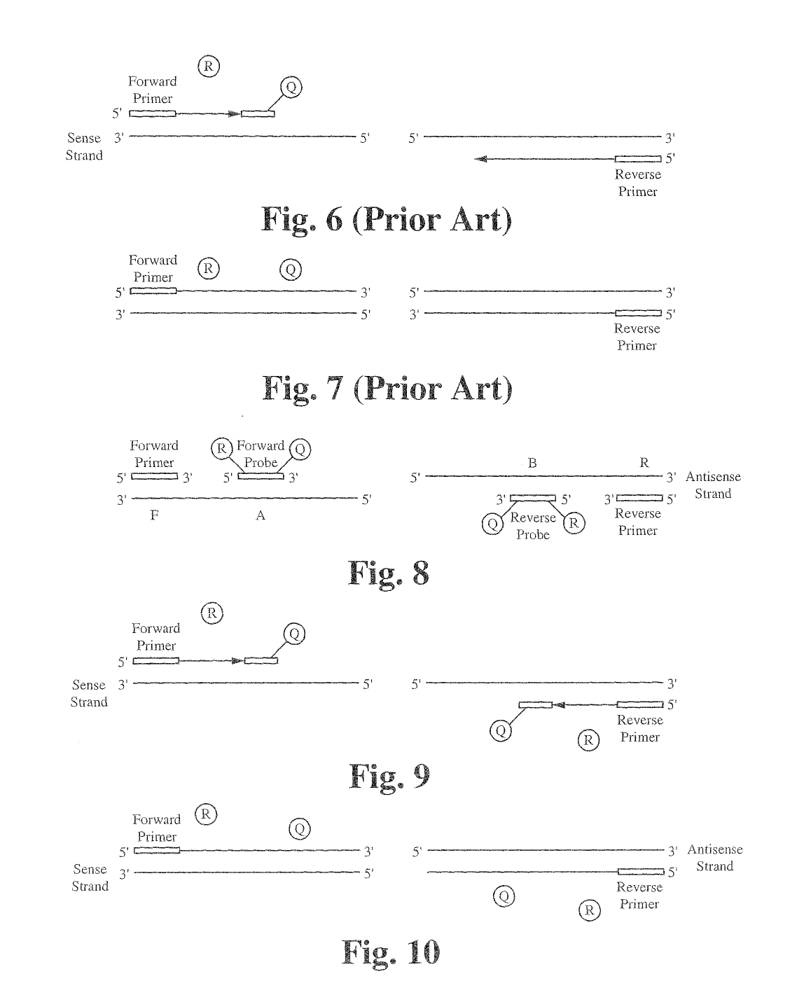

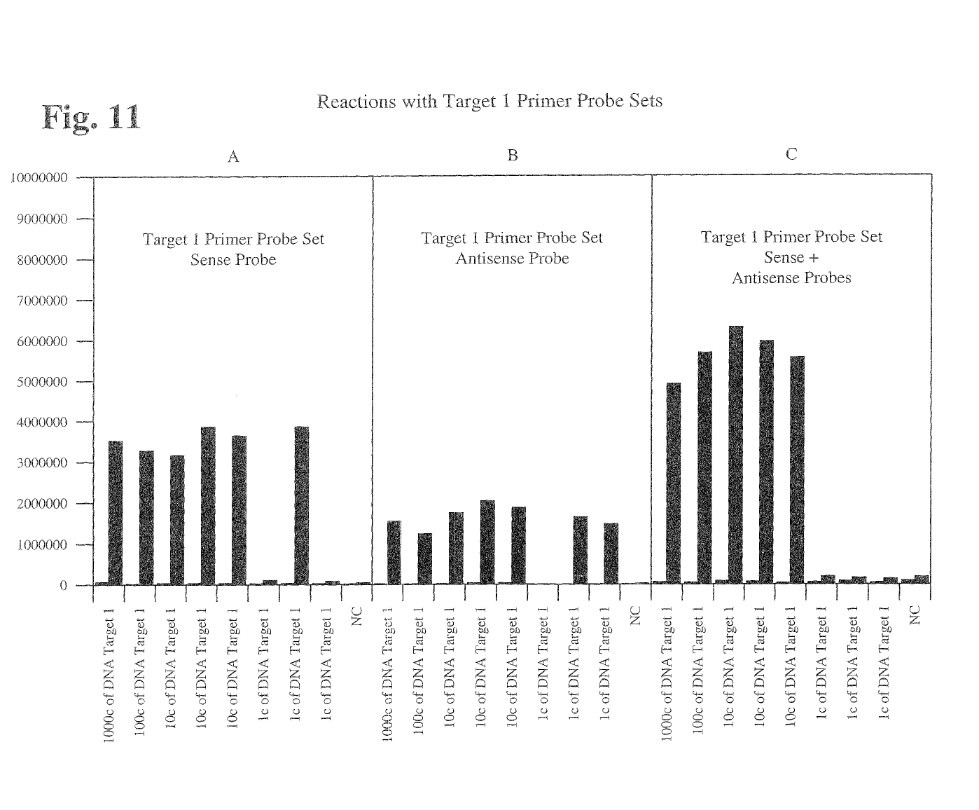

Embodiments of the amplification and detection method are described relative to the several views of the drawings. Where appropriate and only where identical elements are disclosed and shown in more than one drawing, the same reference numeral will be used to represent such identical elements.

### DETAILED DESCRIPTION OF THE PRESENT INVENTION

Reference will now be made in detail to the embodiments of the amplification and detection method, examples of which are illustrated in the accompanying drawings. While the amplification and detection method will be described in conjunction with the embodiments below, it will be understood that they are not intended to limit the amplification and detection method to these embodiments and examples. On the contrary, the amplification and detection method is intended to cover alternatives, modifications and equivalents, which may be included within the spirit and scope of the sample preparation method as defined by the appended claims. Furthermore, in the following detailed description of the amplification and detection method, numerous specific details are set forth in order to more fully illustrate the amplification and detection method. However, it will be apparent to one of ordinary skill in the prior art that the amplification and detection method may be practiced without these specific details. In other instances, well-known methods and procedures, components and processes haven not been described in detail so as not to unnecessarily obscure aspects of the amplification and detection method.

The term “oligonucleotide” as used herein refers to a molecule comprised of two or more nucleotides. Oligonucleotides readily bind to their respective complementary nucleotides, and are therefore used as primers and probes when performing PCR.

The term “polynucleotide” as used herein refers to a chain of nucleotides. In some embodiments, a polynucleotide is artificially made starting from an oligonucleotide, and a polymerase enzyme is used to extend the oligonucleotide by adding nucleotides according to a specified pattern. As applied to PCR, the specified pattern is the template strand of nucleic acid.

The term “primer” as used herein refers to an oligonucleotide which functions as a starting point for strand synthesis. The ologonucleaotide primer is configured to bond to a specific sequence of the template nucleic acid strand. Strand synthesis begins at the primer when placed under conditions that induce synthesis of a primer extension product which is complementary to the target nucleic acid strand. In some embodiments, such conditions exist in the presence of nucleotides and an agent for polymerization, such as DNA polymerase, and at a suitable temperature and pH. In some embodiments, the oligonucleotide primer includes 15-25 or more nucleotides, although it may contain fewer nucleotides.

The term “target sequence” or “target nucleic acid sequence” as used herein, refers to a region of a nucleic acid sequence which is to be either amplified, detected, or both. In some embodiments, the target sequence resides between the two primer sequences used for amplification.

The term “probe” as used herein refers to an oligonucleotide that includes a detectable element, such as a reporter molecule, that is not detectable when still bonded to the nucleic acid sequence of the oligonucleotide, but when released or cleaved from the nucleic acid sequence is detectable. In some embodiments, the 3′ end of the probe is blocked to prohibit incorporation of the probe into a primer extension product. In some embodiments, the detectable element is bonded to the 3′ end of the probe, thereby providing the blocking functionality.

The tenor “detectable element” as used herein refers to any atom or molecule which can be attached to an oligonucleotide, such as a probe, and provides a detectable signal when separated from the oligonucleotide but does not provide a detectable signal while still attached to the oligonucleotide. Detectable elements may provide signals detectable by any conventional technique including, but not limited to, fluorescence, radioactivity, colorimetric, X-ray diffraction or absorption, magnetism, enzymatic activity, and the like.

The term “5′ end to 3′ end nuclease activity” as used herein refers to that activity of a template-specific nucleic acid polymerase including either a 5′ end to 3′ end exonuclease activity associated with some DNA polymerases whereby nucleotides are removed, or cleaved, from the 5′ end of an oligonucleotide in a sequential manner, or a 5′ end to 3′ end endonuclease activity wherein cleavage occurs more than one nucleotide from the 5′ end, or both.

Embodiments of the amplification and detection method are directed to performing nucleic acid amplification and detection using two different types of probes, one probe configured to anneal to a sense strand of a target sequence and another probe configured to anneal to an antisense strand of the target sequence. The amplification and detection method performs real-time PCR using forward primers, forward probes, reverse primers, reverse probes, nucleotides for strand/antistrand extension, and nucleic acid polymerase. Each forward primer is configured to anneal to the 3′ end of the sense strand. The nucleic acid sequence of the 3′ end of the sense strand that is complementary to the forward primer is referred to as sequence F. Each reverse primer is configured to anneal to the 3′ end of the antisense strand. The nucleic acid sequence of the 3′ end of the antisense strand that is complementary to the reverse primer is referred to as sequence R. The forward primer has a different nucleic acid sequence than does the reverse primer, as sequence F has a different nucleic acid sequence than sequence R.

Each forward probe is configured to anneal to a specific sequence on the sense strand, referred to as sequence A. As such, the nucleic acid sequence of the forward probe is complementary to sequence A. Sequence A is an inner sequence to sequence F. In some embodiments, sequence A overlaps with a portion of sequence F. The forward probe includes a detectable element and the forward probe is configured to suppress the detectable characteristic of the detectable element while the detectable element is attached to the forward probe. In some embodiments, the detectable element is a reporter molecule and the forward probe also includes a quencher molecule configured to suppress the detectable characteristic of the quencher molecule while both are attached to the forward probe, as is well known in the art. In some embodiments, the reporter molecule is a fluorescent dye that radiates a known wavelength of light. In other embodiments, the reporter molecule is any conventional element that can be attached to the forward probe and has a detectable characteristic that can be suppressed or changed while attached to the forward probe, but that can be detected when detached or released from the forward probe.

Each reverse probe is configured to anneal to a specific sequence on the antisense strand, referred to as sequence B. As such, the nucleic acid sequence of the reverse probe is complementary to sequence B. Sequence B is an inner sequence to sequence R. In some embodiments, sequence B overlaps with a portion of sequence R. The reverse probe includes a detectable element and the reverse probe is configured to suppress the detectable characteristic of the detectable element while the detectable element is attached to the reverse probe. In some embodiments, the detectable element is a reporter molecule and the reverse probe also includes a quencher molecule configured to suppress the detectable characteristic of the quencher molecule while both are attached to the reverse probe. In some embodiments, the reporter molecule is a fluorescent dye that radiates a known wavelength of light. In other embodiments, the reporter molecule is any conventional element that can be attached to the reverse probe and has a detectable characteristic that can be suppressed or changed while attached to the reverse probe, but that can be detected when detached or released from the reverse probe.

In some embodiments, the detectable element of the forward probe is the same as the detectable element of the reverse probe. For example, the detectable element for both the forward probe and the reverse probe is a fluorescent dye, such as ROX dye or VIC dye. It is understandable that any conventional fluorescent dye can be used as the detectable element. An objective of the amplification and detection method is to determine if the target nucleic acid sequence is present in the original sample. This determination is made by detecting the presence of the amplified product. The presence of either the sense strand or the antisense strand corresponding to the target nucleic acid enables this determination. Therefore, it is not an objective to differentiate the sense strand from the antisense strand, which necessitates the use of a different detectable element for the forward probe that for the reverse probe. Instead, where such a distinction is not required, using the same detectable element for both the sense strand and the antisense strand provides an additive effect having advantageous results. One advantage is that a stronger overall signal is provided for the same number of thermal cycles. Another advantage is an increase in the signal to noise ratio which lowers the limits of detection.

The forward probe has a different nucleic acid sequence than does the reverse probe, as sequence A has a different nucleic acid sequence than sequence B. As such, there is no competition between the forward probe and the reverse probe to anneal to the same complimentary sequence. By annealing to different sequences, the forward probe and the reverse probe complement each other.

The target nucleic acid sequence that is to be amplified and detected is included in an original sample. In its original state, the target nucleic acid sequence is either a single strand of nucleic acid or a double strand. If the original sample includes double-stranded target nucleic acid sequence, then the sense strand and the antisense strand are present in the original sample, the sense strand and the antisense strand being the two strands of the original double-stranded target nucleic acid sequence. If the original sample includes single-stranded target nucleic acid sequence, then the original sample includes only the sense strand or the antisense strand. After a first thermal cycle during which the single strand is used as a strand template for strand extension, the resulting double stranded product includes the sense strand and the antisense strand. Once melted during the denaturing step of the thermal cycle, the separated sense strand and antisense strand are subsequently used as templates for extension.

FIGS. 8-10 illustrate a nucleic acid amplification and detection method using PCR according to an embodiment. A sense strand and an antisense strand of a target nucleic acid sequence to be amplified are shown. During each thermal cycle of the nucleic acid amplification process, the sense and antisense strands are separated during the heating, or denaturing, step of the cycle, as shown in FIG. 8. The separated strands then function as templates during the subsequent cooling, or annealing, step of the thermal cycle. Prior to the thermal cycling steps, PCR reagents are added to the target sequence to be amplified. The PCR reagents include forward primers, forward probes, reverse primers, reverse probes, nucleotides for strand/antistrand extension, and nucleic acid polymerase. The forward primers and the reverse primers are collectively referred to as primer pairs. Each forward primer is complementary to the 3′ end of the sense strand, and therefore anneals to sequence F on the 3′ end of the sense strand during the annealing step, as shown in FIG. 8. Similarly, each reverse primer is complementary to the 3′ end of the antisense strand, and therefore anneals to sequence R on the 3′ end of the antisense strand during the annealing step, as shown in FIG. 8. Each of the reverse probes and the forward probes are an oligonucleotide including a detectable element. In the application shown in FIGS. 8-10, the detectable element is a reporter molecule on the 5′ end of the probe, and each probe also includes a quencher molecule on the 3′ end. It is understood that the forward probes and the reverse probes can be configured with detectable elements and suppression mechanisms other than reporter/quencher molecule pairs. Although the nucleic acid sequence of the forward probe is different than the nucleic acid sequence of the reverse probe, the reverse probe and the forward probe each include the same detectable element.

Each forward probe is complementary to an inner sequence A of the sense strand, and each reverse probe is complementary to an inner sequence R of the antisense strand. During the annealing step, a forward probe anneals to the inner sequence A of the sense strand, which is downstream, e.g. in the direction of strand extension, from the annealed forward primer, as shown in FIG. 8. While both the reporter molecule and the quencher molecule remain bound to the forward probe, either in solution or annealed to the sense strand, the reporter molecule is not detectable because of the proximity of the quencher molecule. Also during the annealing step, a reverse probe anneals to the inner sequence B of the antisense strand, which is downstream, e.g. in the direction of strand extension, from the annealed reverse primer, as shown in FIG. 8. While both the reporter molecule and the quencher molecule remain bound to the reverse probe, either in solution or annealed to the antisense strand, the reporter molecule is not detectable because of the proximity of the quencher molecule.

During an extension step of the thermal cycle, a 5′ end to 3′ end strand extension, or polymerization, of both the sense strand and the antisense strand is performed where complimentary nucleotides anneal in sequence according to the strand template and the antistrand template. FIG. 8 illustrates the sense strand and the antisense strand at the onset of polymerization. Strand extension begins from the forward primer annealed to the 3′ end of the sense strand and progresses toward the 5′ end of the sense strand. Similar strand extension begins on the antisense strand beginning from the reverse primer annealed to the 3′ end of the antisense strand.

During the extension step, the forward probe annealed to the sense strand is digested by 5′ end to 3′ end nuclease activity of the polymerase, and the reverse probe annealed to the antisense strand is also digested by 5′ end to 3′ end nuclease activity. FIG. 9 illustrates the 5′ end of the forward probe being cleaved and the 5′ end of the reverse probe being cleaved, thereby releasing the reporter molecules from each of the forward probe and the reverse probe. FIG. 10 illustrates completion of the polymerization process including digestion of the entire forward probe previously annealed to the sense strand and the entire reverse probe previously annealed to the antisense strand. Upon digestion, the quencher molecule is no longer close enough to the reporter molecule to quench emissions by energy transfer. As more and more forward and reverse probes are digested during the thermal cycling amplification process, more and more of the reporter molecules are released, resulting in a stronger and stronger fluorescent signal.

A number of thermal cycles must be performed before a sufficiently strong signal is generated for detection. As compared to conventional real-time PCR methods, the number of thermal cycles needed to reach a detection threshold, referred to as a threshold cycle, is reduced, as the amount of released reporter molecules for any given number of thermal cycles is essentially doubled that of the conventional methods. The threshold cycle for any given sample depends on the amount of target nucleic acid sequence originally present in the sample. The greater the original amount of target nucleic acid sequence, the lower the threshold cycle. In real-time PCR detection, the detectable signal is measured once every thermal cycle, typically during the annealing step. Any conventional detection technique can be used. In some embodiments, a light source and sensor are used to direct light into the sample solution and measure the resulting light received by the sensor.

FIG. 11 illustrates signal strength test results measured at the end of PCR, referred to as end point PCR, for a first exemplary single-plea target nucleic acid sequence. The forward probes and the reverse probes used in the amplification process applied to the first target nucleic acid sequence are configured with ROX dye and the reporter molecule. Three columns A, B, and C are included in FIG. 11. Column A measures the results when only forward probes are included in the PCR reagents. The reverse probes are not included in the test results shown in the left chart. Column B measures the results when only reverse probes are included in the PCR reagents. The forward probes are not included in the test results shown in column B. Column C measures the results when both forward probes and reverse probes are included in the PCR reagents.

Each bar in the column represents the end of PCR results for a separate test. The height of each bar represents the measured signal strength at the end of the PCR process. From left to right, the first bar represents test results from an original test sample having 1000 copies of the target nucleic acid sequence. The second bar represents test results from an original test sample having 100 copies of the target nucleic acid sequence. The third, fourth, and fifth bars represent test results from original test samples each having 10 copies of the target nucleic acid sequence. The sixth, seventh, and eighth bars represent test results from original test samples each having 1 copy of the target nucleic acid sequence. The ninth bar represents test results from an original test sample having zero copies of the target nucleic acid sequence. The ninth test is performed as a control, which should result in zero detectable signal.

As shown in FIG. 11, each bar includes a left bar and a right bar pair. In practice, the reporter molecule in each probe is not 100% quenched while attached to the probe. As such, there is a residual fluorescence signal, also referred to as a background fluorescence, detected even in reactions that have not undergone any amplification, and therefore have not had enzyme facilitated 5′ end to 3′ end exonuclease activity. In end point PCR, the fluorescence of the reaction is first measured in the beginning of the reaction, before PCR has commenced, thereby measuring the background fluorescence. The left bar in the bar pair is a measure of this beginning point measurement. The fluorescence of the reaction is again measured at the end of the reaction upon completion of the PCR process. The right bar in the bar pair is a measure of this end point measurement. The difference between the fluorescence before and after the PCR process is deterministic of amplification and hence a positive detection.

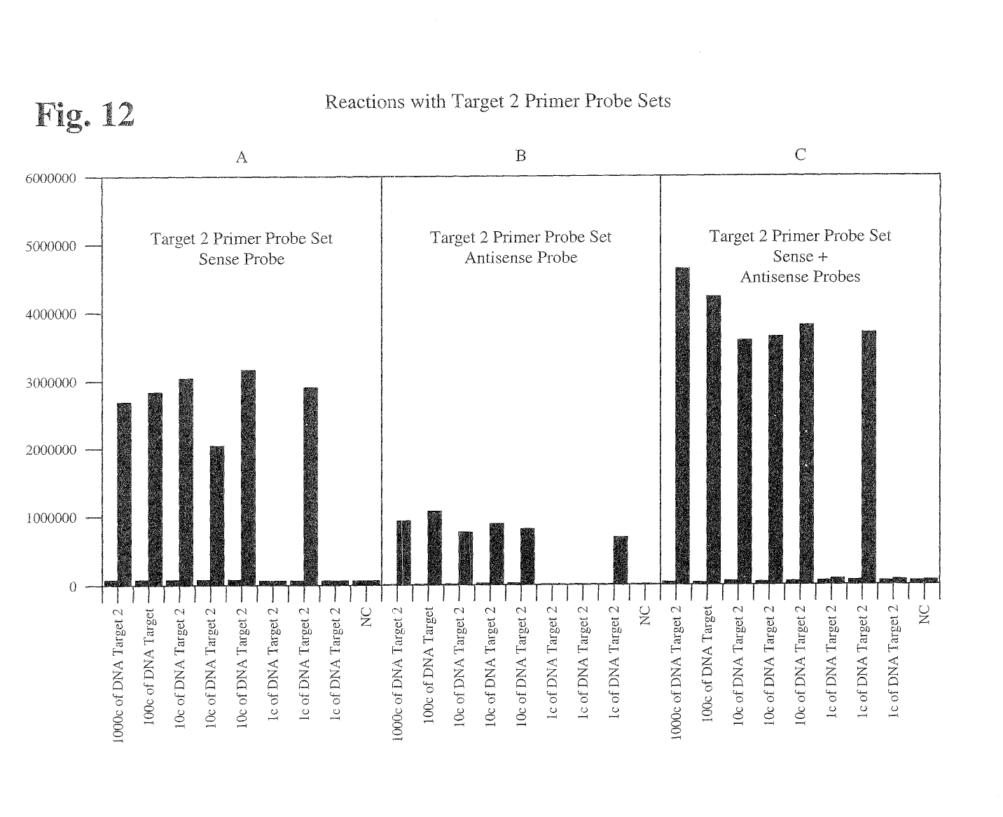

FIG. 12 illustrates signal strength test results measured at the end of PCR for a second exemplary single-plex target nucleic acid sequence. The forward probes and the reverse probes used in the amplification process applied to the second target nucleic acid sequence are configured with VIC dye and the reporter molecule. Three columns A, B, and C are included in FIG. 12, similar in context as the three columns in FIG. 11. Column A measures the results when only forward probes are included in the PCR reagents. The reverse probes are not included in the test results shown in column A. Column B measures the results when only reverse probes are included in the PCR reagents. The forward probes are not included in the test results shown in column B. Column C measures the results when both forward probes and reverse probes are included in the PCR reagents.

As can be seen in column C in both FIGS. 11 and 12, the signal strength is significantly greater when both forward probes and reverse probes are used. As shown in both FIGS. 11 and 12, the antisense probes are not as efficient as the sense probes. In other words, the fluorescence generated from the antisense probes is not as strong as that generated by the sense probes. This difference provides a means for discriminating between detection of the sense strand and the antisense strand if desired. If strand level discrimination is not desired, the antisense probe and the sense probe can be designed to generate the same or similar fluorescence strength. Further, the results shown in FIGS. 11 and 12 indicate that the use of both forward probes and reverse probes results in an additive effect. The results for those tests starting with 10 copies or more of the target nucleic acid sequence consistently show increased signal strength. For those tests starting with 1 copy, a signal is not always detected, but when it is, as in FIG. 12, the result is again an increased signal strength.

The amplification and detection method is described above as using probes that include reporter and quencher molecules for generating and suppressing a detectable signal identifying the presence of the target nucleic acid sequence. It is understood that the probes can utilize alternative detectable elements that are detectable when released from the probes, but not detectable while still attached to the probes. Examples of such alternate detectable elements include, but are not limited to, enzyme-based detection methods in combination with Biotinylated oligonucleotides, radionucleid labeled probes. Detectable elements may provide signals detectable by any conventional technique including, but not limited to, fluorescence, radioactivity, colorimetric, X-ray diffraction or absorption, magnetism, enzymatic activity, and the like.

The amplification and detection method is described above as using the same detectable element for both the sense and antisense probes, resulting in a cumulative detectable signal. In alternative embodiments, the detectable element of the sense probe is different than the detectable element of the antisense strand, different in the context that the detectable element of the sense strand is uniquely identifiable from the detectable element of the antisense strand. For example, the detectable element of the sense strand is a first dye, and the detectable element of antisense strand is a second dye. The first dye and the second dye each emit a different wavelength of light sufficiently different in wavelength as to be uniquely detected and identified. The emitted light is detected, and based on the identified wavelength of the detected light, the type of dye is determined. In this respect, the first dye and the second dye are each uniquely detectable and identifiable. Although using uniquely identifiable detectable elements for the sense and antisense probes negates the cumulative detectable signal aspect of the amplification and detection method, using uniquely identifiable detectable elements for the sense and antisense probes does allow for independent identification of the sense strand and the antisense strand, and for diagnosing differences between the sense strand and the antisense strand.

The amplification and detection method has been described in terms of specific embodiments incorporating details to facilitate the understanding of the principles of construction and operation of the amplification and detection method. The specific configurations shown and the methodologies described in relation to the various modules and the interconnections therebetween are for exemplary purposes only. Such reference herein to specific embodiments and details thereof is not intended to limit the scope of the claims appended hereto. It will be apparent to those skilled in the art that modifications may be made in the embodiment chosen for illustration without departing from the spirit and scope of the amplification and detection method.

What is claimed is:

1. A method of detecting nucleic acid amplification, the method comprising:
a. performing a thermal cycling process on a fluid sample including a target nucleic acid sequence to be amplified, nucleic acid polymerase, a forward primer, a forward probe, a reverse primer, and a reverse probe, wherein during an annealing step of the thermal cycling process the forward primer is configured to anneal to a first complementary sequence of a sense strand of the target nucleic acid sequence, the forward probe is configured to anneal to a second complementary sequence of the sense strand, the reverse primer is configured to anneal to a first complementary sequence of an antisense strand of the target nucleic acid sequence, and the reverse probe is configured to anneal to a second complementary sequence of the antisense strand,
the forward probe includes a detectable element that is not detectable when attached to the forward probe and is detectable when separated from the forward probe,
the reverse probe includes the detectable element that is not detectable when attached to the reverse probe and is detectable when separated from the reverse probe,
wherein during an annealing step of the thermal cycling process, a strand extension is performed on the sense strand and the antisense strand using the nucleic acid polymerase, thereby cleaving the forward probe and the reverse probe which separates the detectable element from the forward probe and the detectable element from the reverse probe; and
b. detecting the separated detectable elements.

2. The method of claim 1 wherein the first complementary sequence of the sense strand comprises a 3′ end of the sense strand.

3. The method of claim 1 wherein the first complementary sequence of the sense strand and the second complementary sequence of the sense strand are completely separate sequences.

4. The method of claim 1 wherein the first complementary sequence of the sense strand and the second complementary sequence of the sense strand partially overlap.

5. The method of claim 1 wherein the first complementary sequence of the antisense strand comprises a 3′ end of the antisense strand.

6. The method of claim 1 wherein the first complementary sequence of the antisense strand and the second complementary sequence of the antisense strand are completely separate sequences.

7. The method of claim 1 wherein the first complementary sequence of the antisense strand and the second complementary sequence of the antisense strand partially overlap.

8. The method of claim 1 wherein the forward probe further includes a suppressing element that suppresses detection of the detectable element when both the detectable element and the suppressing element are attached to the forward probe, the reverse probe further includes the suppressing element that suppresses detection of the detectable element when both the detectable element and the suppressing element are attached to the reverse probe.

9. The method of claim 8 wherein the detectable elements comprises a reporter molecule and the suppressing element comprises a quencher molecule.

10. The method of claim 1 wherein the strand extension comprises a 5′ end to 3′ end strand extension.

11. The method of claim 1 wherein the second complementary sequence of the sense strand is different than the second complementary sequence of the antisense strand.

12. The method of claim 1 wherein an original sample of the target nucleic acid sequence is single-stranded.

13. The method of claim 1 wherein an original sample of the target nucleic acid sequence is double-stranded.

14. The method of claim 1 wherein performing the thermal cycling process comprises performing a polymerase chain reaction amplification process.
